(19) United States
(12) Patent Application Publication
(10) Pub. No.: US 2026/0232896 A1
(43) Pub. Date: Aug. 13, 2026

(54) ENHANCED PATCH PUMP SET UP

(71) Applicant: Beta Bionics, Inc., Irvine, CA (US)

(72) Inventors: David Lim, Irvine, CA (US); Mike Mensinger, San Diego, CA (US); Himanshu Patel, Concord, MA (US); Sean Saint, San Diego, CA (US)

(73) Assignee: Beta Bionics, Inc., Irvine, CA (US)

(21) Appl. No.: 19/536,451

(22) Filed: Feb. 11, 2026

Related U.S. Application Data
(60) Provisional application No. 63/756,880, filed on Feb. 11, 2025.
(60) Provisional application No. 63/786,397, filed on Apr. 10, 2025.

(51) Int. Cl. A61M 5/142; A61M 5/168; A61M 5/172

(52) U.S. Cl. CPC A61M 5/14248; A61M 5/16804; A61M 5/1723; A61M 2005/14252

(57) ABSTRACT

Embodiments of an enhanced PATCH PUMP system leverage cognitive load free set up and enable users to have form factors and priming based on body weight with no other set up or data needed to use systems which likewise host further combination of patch pumps and algorithmic base schematics.

### CROSS REFERENCE TO RELATED CASES

The present application claims priority to U.S. Provisional Application No. 63/756,880, filed Feb. 11, 2025, and to U.S. Provisional Application No. 63/786,397, filed Apr. 10, 2025, the contents of which are incorporated herein by reference in their entirety.

### BACKGROUND OF THE DISCLOSURES AND INVENTIONS

### Technical Field

This disclosure relates to improved blood glucose control systems and to patch pump types of ambulatory medical devices that provide therapy to a subject. Such devices have evolved, from the INSULET/omnipod types of approaches to now include combinations of durables and disposables, with generified mechanical and infusion elements imparting better and better algorithmic kinds of logic delivery schemes to manage therapy for diabetics with less users inputs and interactions then originally thought to be needed Likewise, any insulin and/or glucagon or bihormonal based monitoring, managing and or fluid delivery systems for treating diabetes driven by algorithms are implicated by the teachings of the present invention and only serves to further underscore novelties of the instant approaches relative to the use of glucose level control systems, including excursions in these processes, which remain challenging and are needing to be impactful given the ongoing growth of this set of disease states. Those skilled in the art likewise embrace known terms, abbreviations and standard scientific notations employed throughout this document, and/or expressly spelled out in the Appendix as used by skilled artisans, engineers, scientists thus not further defined in this summary.

### Description of Related Art

Sustained delivery, pump driven medicament injection devices, of the patch pump variety are embodied in disposable and durable device components and/or those means for delivery to the ever-improving variable nature of insulin/glucagon and the like medicaments to patients, and likewise highly impacted by the individuated costs of goods, these systems generally include a delivery cannula mounted in a subcutaneous manner through the skin of the subject at an infusion site. The pump draws medicine from a reservoir and delivers it to the subject via the cannula. The injection device typically includes a channel that transmits a medicament from an inlet port to the delivery cannula which results in delivery to the subcutaneous tissue layer where the delivery cannula terminates. Some infusion devices are configured to deliver one medicament to a subject while others are configured to deliver multiple medicaments to a subject.

Likewise, Artisans understand that management of qualitative meal announcements and algorithm detected meals by types of meals and glucose values and parameters over time remains the cutting edge of this treatment area and attempts to balance diabetic disease states.

Traditional meal boluses are delivered, for example, based on number of carbohydrates [used interchangeably with “carb(s)” herein] to be consumed, a patients'insulin to carb ratio, starting glucose and glucose target. One challenge in this is that it is hard to estimate the number of carbs plus getting the true value of insulin to carb ratio is difficult. An approach generating dosages including post-prandial glucose excursions and respective values and comparisons has yet to be fully developed among known systems. Herein, blood glucose control systems and ambulatory medical devices that provide therapy to a subject, such as blood glucose control, are disclosed. Disclosed systems and devices can implement one or more features that improve functionality by establishing post-prandial data sets, inter alia, and evaluation periods to adjust meal boluses up or down.

Longstanding needs in these fields require ongoing and constant forays into new territory for making better dosed and more highly functional systems given the learning that such variables may be directly relevant to peaks and valleys inherent in their management.

Likewise, those skilled in the art have become aware that insulin sensitivity and glucose level control need to be re-assessed to drive optimal dosing needs, based upon individuated tolerance levels and likely requires moving dosage response therapy to the next level, from basal titration to what responses are to needed to optimally solve for carbohydrate to glucose levels—it is respectfully proposed that data from meal adaptations to post-prandial excursions is believed to have significant impacts in these fields, which need to be adapted in real time by the closed loop systems being offered herein for consideration.

### OBJECTS & SUMMARY OF THE INVENTIONS

Briefly stated, new methods for adapting patch pumps to the developed patients and users needs are identified. Additionally, in a bihormonal implementation, the meal dose may be further adapted based on the amount of glucagon delivered for example reducing the meal size if glucagon is delivered.

During the management of everything from glucose excursions activities are monitored and the meal bolus associated with this qualitative announcement is increased or decreased if the glucose values or pattern is higher or lower than expected, including improved algorithmic derivations of these data sets and glucose values.

According to embodiments, there are provided patch pump systems and methods whereby a user enters a qualitative meal announcement (for example B, L, D+S, M, L) or a algorithmically detected meal. The system initially gives a meal bolus which is conservative and based on rules of thumb, such as based on patient demographics e.g. body mass or TDD. During the post-prandial period, the glucose excursions are monitored and the meal bolus associated with this qualitative announcement is increased or decreased if the glucose values or pattern is higher or lower than expected.

According to embodiments, there are provided patch pump systems which titrate meal doses based at least in part on post-prandial glucose excursions, further comprising an ambulatory medicament device configured to generate a dose control signal for delivery of medicament to a subject, the ambulatory medicament device comprising, a medicament delivery interface configured to operatively connect to a medicament pump for infusing medicament into the subject; a display interface configured to output display signals configured to generate user interface screens on a display device; a memory configured to store specific computer-executable instructions; and a hardware processor in communication with the memory and configured to execute the specific computer-executable instructions to at least: generate the dose control signal using a control algorithm employing control parameters, wherein at least one control parameter of the control parameters is driven by meal adaptations using post-prandial insulin evaluation.

According to embodiments, there are disclosed methods for delivering with patch pumps improved approaches to adapting meals using post-prandial insulin, comprising, in combination, delivering a meal bolus and establishing a post-prandial evaluation period; estimating an expected or an ideal total glucose deviation based on meal size and insulin dose; comparing actual total glucose to expected value; ranking minimum glucose and glucose at end of period; and adjusting meal boluses up or down. Likewise, there are disclosed methods for adapting meals using post-prandial insulin comprising, in combination, delivering a meal bolus and establishing a post-prandial evaluation period; evaluating glucose levels during this period; comparing actual total glucose to expected values; potentially replacing traditional bolus calculations in whole or in part; and adjusting meal boluses up or down.

According to embodiments, there are disclosed durable and disposable patch pump combinations for methods of determining if meal dose or correction dose or basal rate needs to be adjusted, driven by an algorithm according to at least the following steps, if a user doesn't announce a meal, system detects meals automatically and doses for a meal; wherein a user announces a meal generally (no meal type or size); wherein there is a fixed dose—user announces type of meal (e.g. quantified by category such as breakfast, lunch, dinner, snack, and the like); said meal size estimation being at least one of: small, medium, large, extra large, and the like, including Meal Type and Size (for example—Small and Breakfast); Carb entry and/or encompassing values for a Specific food—for example—pizza, ice-cream and the like. In the case of the user does not announce a meal, the system may detect the meal automatically and classify the meal based on time of day or the glucose response of the meal or other system detected characteristics. This class of meals may be adapted using the techniques described herein. This classification may include indication of faster or slower acting carbs in addition to indications of the total number carbs consumed in the meal.

### BRIEF DESCRIPTION OF THE DRAWINGS

The systems, methods, and devices of this disclosure each have more than several innovative aspects, no single one of which is solely responsible for all of the desirable attributes disclosed herein. Details of one or more implementations of the subject matter described in this specification are set forth in the accompanying drawings and the description below, which are merely illustrative and not limiting, often omitting state of the art details for clarity of description, as known to those having a modicum of skill in the art.

FIG. 1 shows a cartooned schematic of an embodiment of a patch pump functioning as part of a patch pump and the essential elements of the same.

FIG. 2 shows a cartooned schematic of an example blood glucose control system embodied in a patch pump set up namely prepare a syringe and remove end cap.

FIG. 3 shows a cartooned schematic of an example blood glucose control system embodied in a PPs.

FIG. 4 shows a cartooned schematic of an example blood glucose control system embodied in a patch pump, namely.

FIG. 5 shows a cartooned schematic of an example blood glucose control system embodied in a patch pump, for set up the step of filling a syringe with medicament.

FIG. 6 shows a cartooned schematic of an example blood glucose control system embodied in a set up procedure namely to release air bubbles.

FIG. 7 shows a cartooned schematic of an example blood glucose control system embodied in a patch pump according to the instant system to fill a cartridge.

FIG. 8 shows a cartooned schematic of an example blood glucose control system embodied in a patch pump, namely the step of scanning or entering a QR code.

FIG. 9 shows a cartooned schematic of the final assembly step—namely to assemble the patch pump namely to slide the pump and filled cartridge together until there is an audible click.

FIG. 10 illustrates a partial plan view of an example ambulatory medical device being an enhanced patch pump system being primed prior to attachment.

FIG. 11 illustrates a dorsal view shown where on a user's body to clean before attachment of sections removable for a combination of durable and disposable assembly view of the ambulatory medical device shown manifested in an enhanced patch pump configuration.

FIG. 12 shows a final step in the patch pump set up process, namely removal of a pull tab and liner assembly.

### DETAILED DESCRIPTIONS OF PREFERRED EMBODIMENTS

The present inventors have discovered and herein disclose enhanced patch pump set up with virtually no cognitive load and fill and snap together and prime with body weight being the only requirements.

Such novelties driven by a new paradigm whereby patch pumps embody all of the features embodied in the software and logic structures emplaced within and transmitted to extant hardware developed for adaptive learning, with simplified and generified access to third party systems.

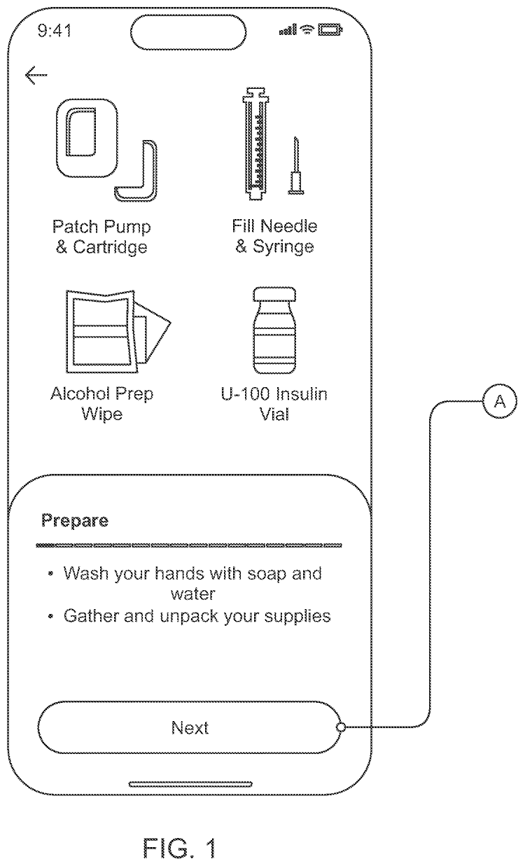

FIG. 1 though 12 are self-explanatory, albeit fundamentally novel and enhanced in that the entire process can be completed with almost no cognitive load.

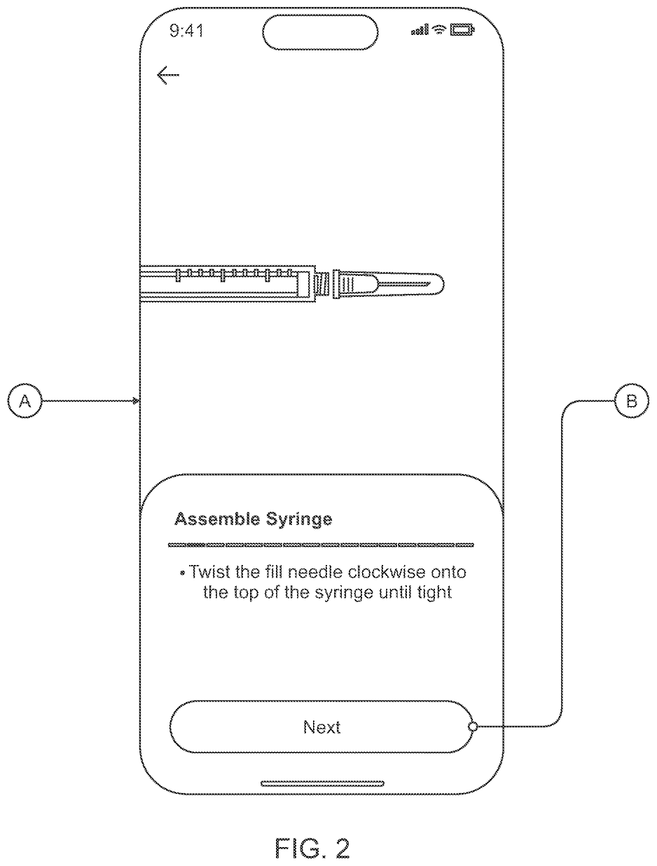

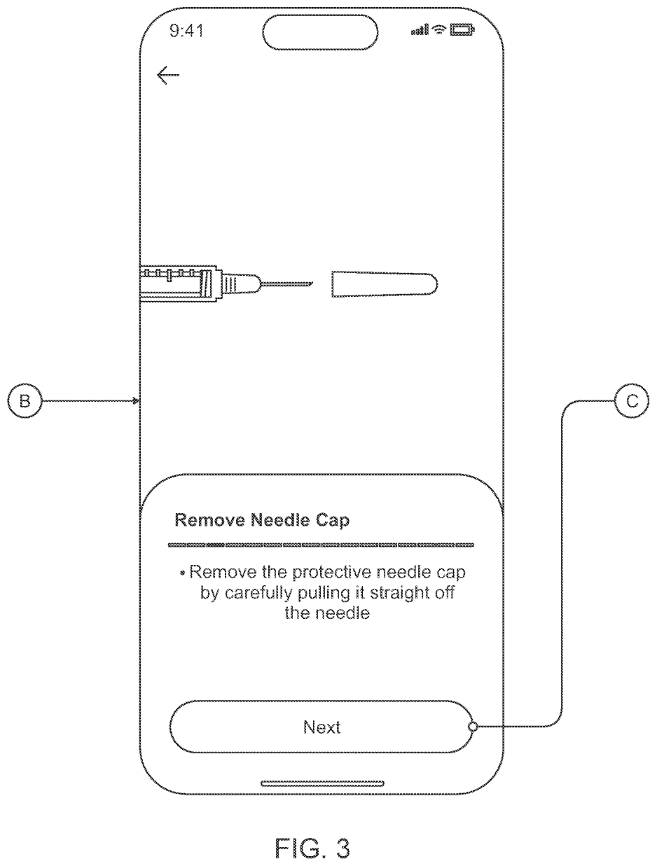

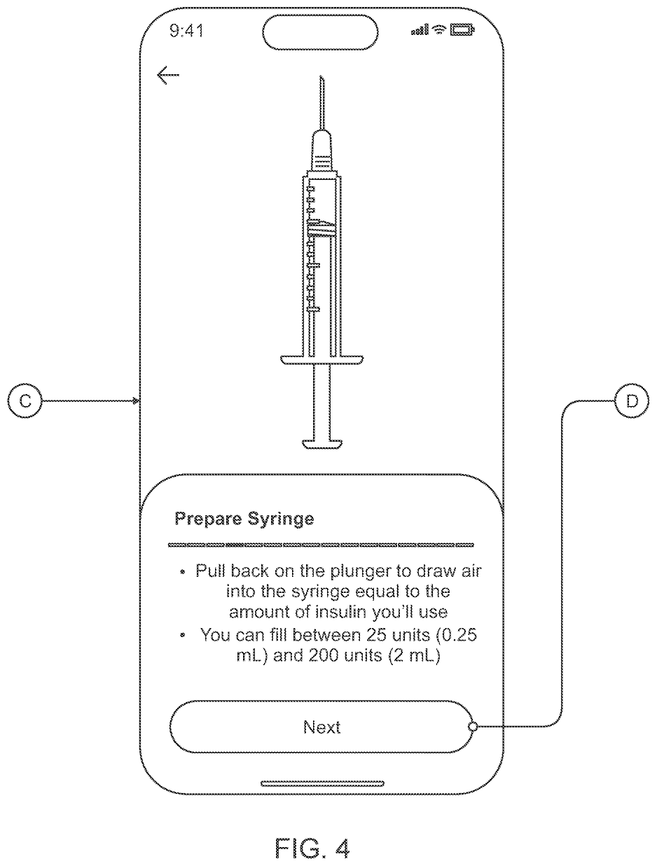

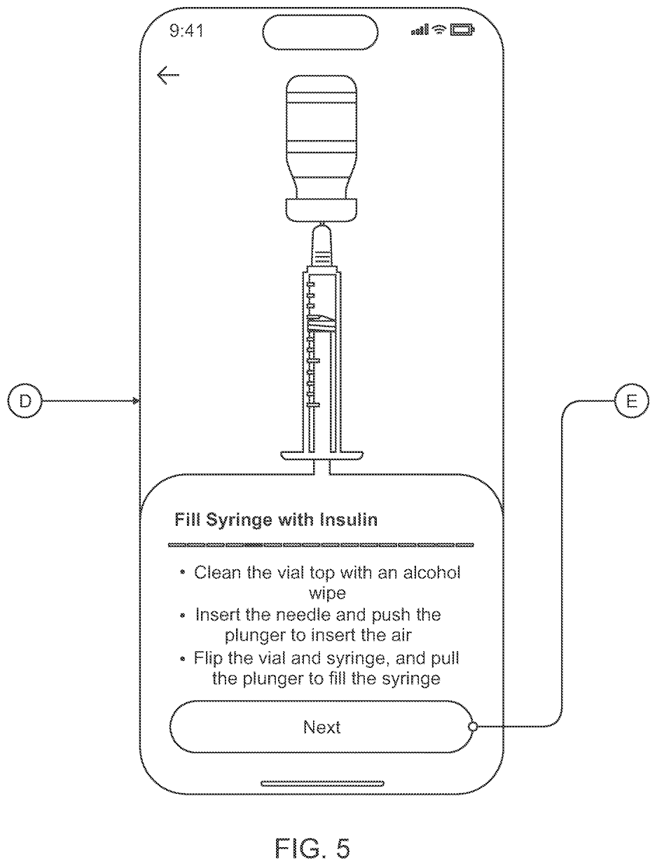

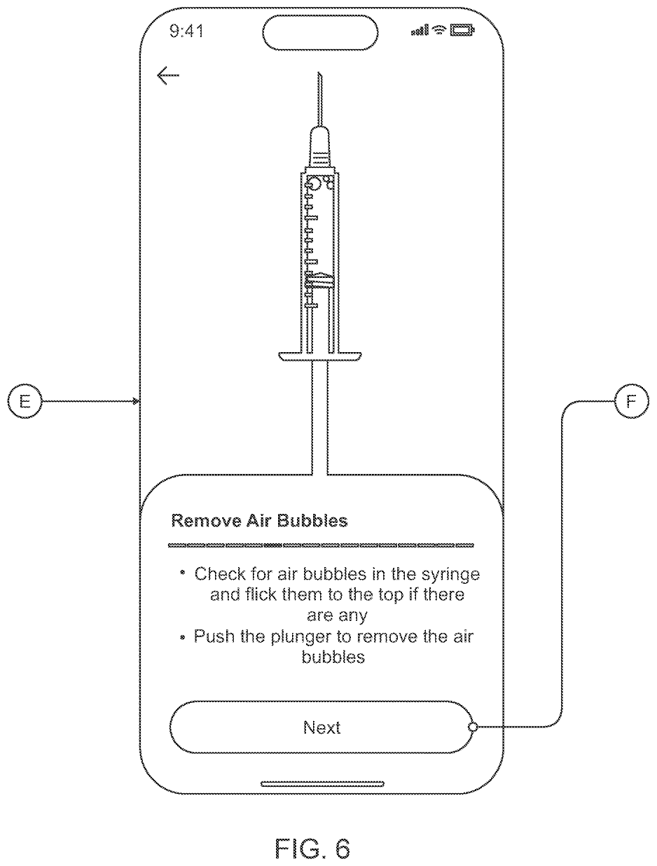

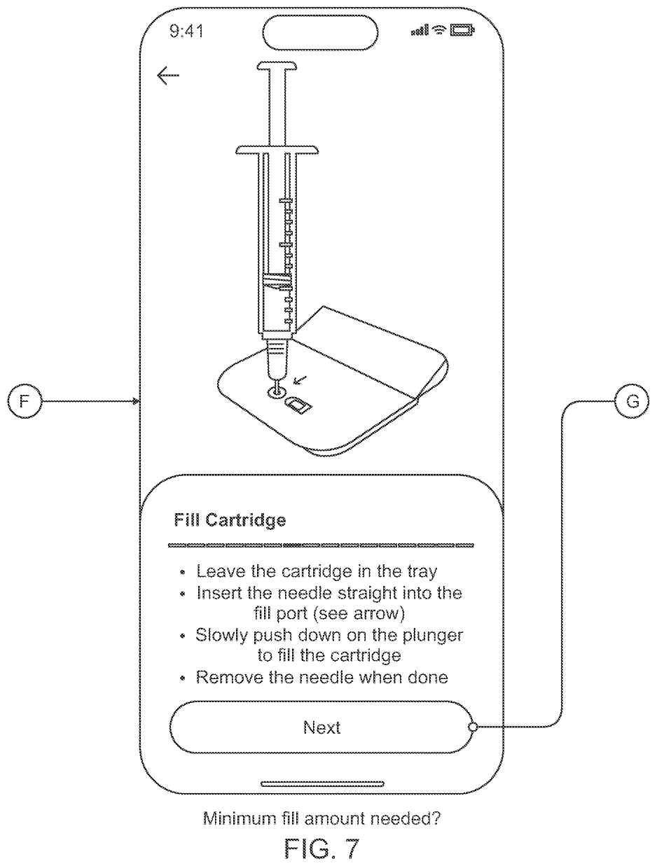

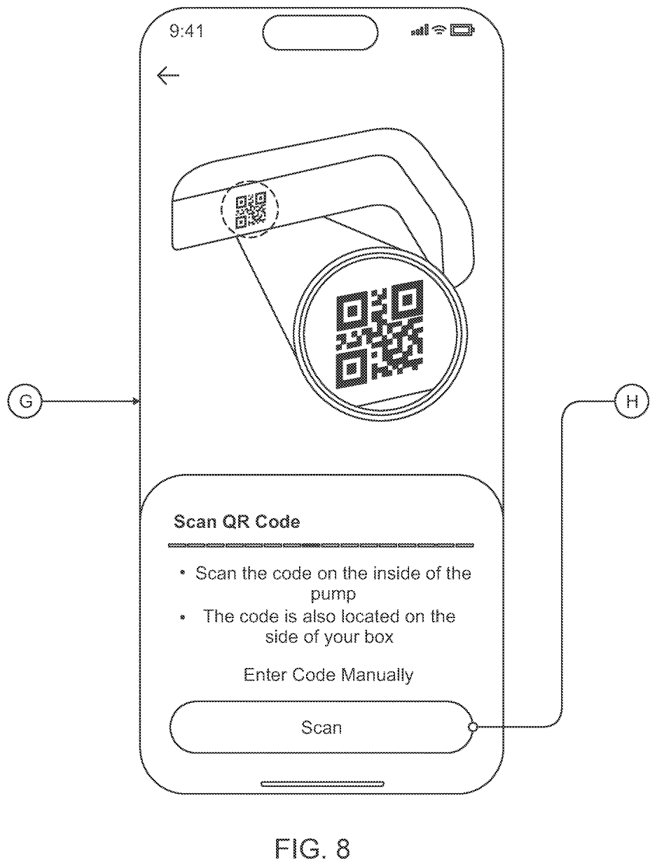

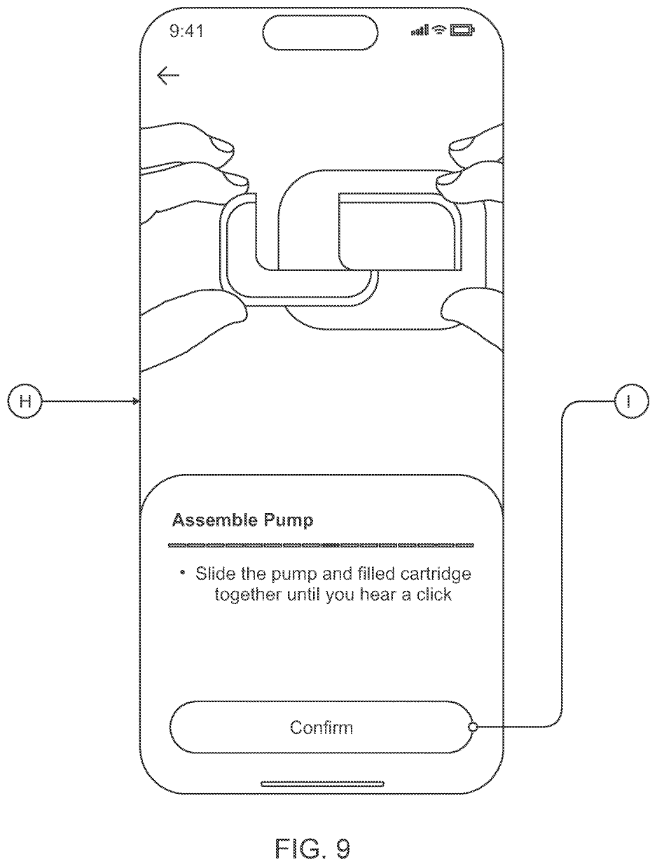

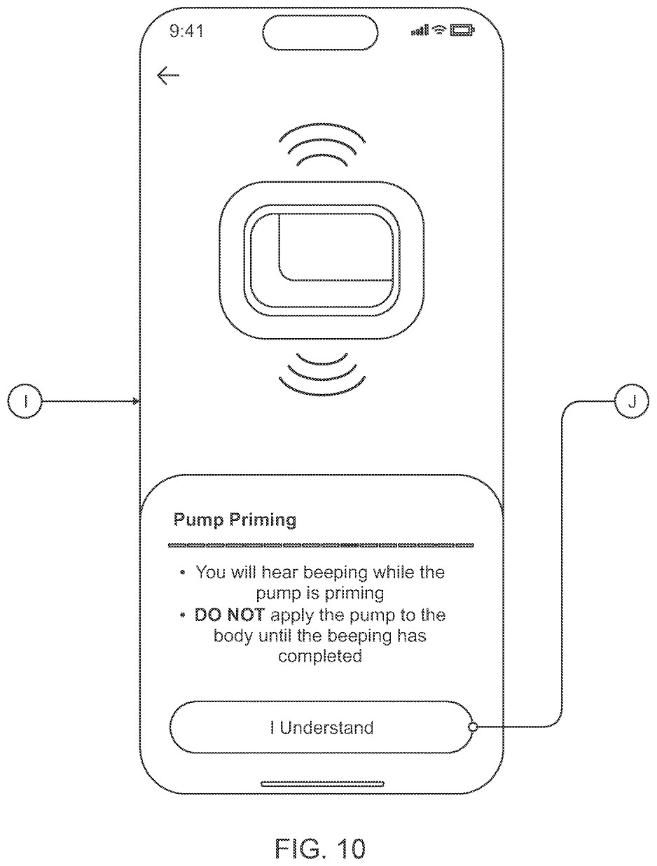

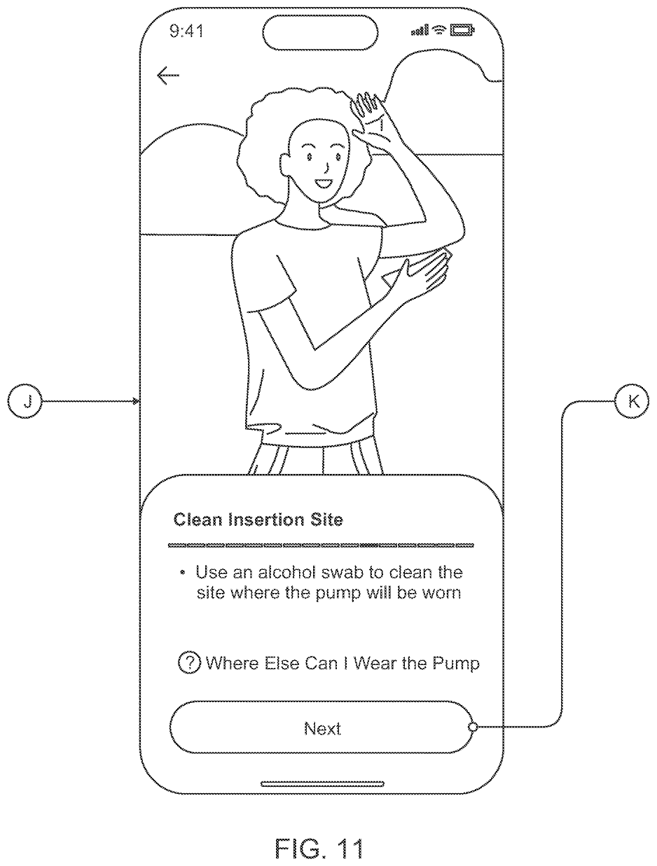

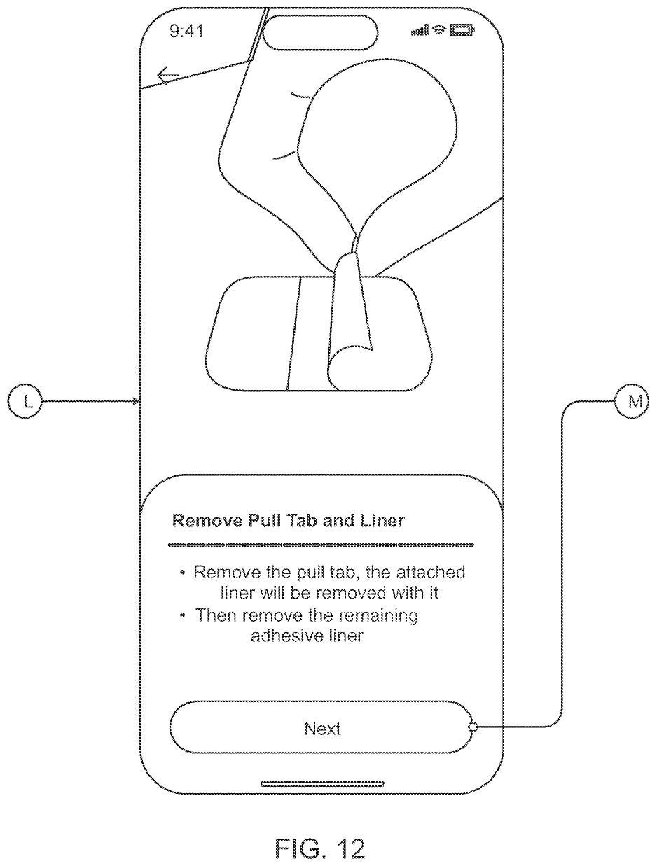

A user requires no training to assemble, prime and attach a working patch pump according to the instant system. FIG. 1 shows the essential elements, namely the durable and disposable elements of the instant patch pumps, along with fill needles, syringes and medicaments. FIG. 2-5 show the steps assemble syringes, remove needle caps and fill with medicaments the instant patch pumps. FIG. 6-12 complete the set up process with almost zero cognitive load to prime, accurate and attach the instant patch pumps

Likewise integrated herein as previously disclosed and further explained in copending U.S. Ser. No. 63/768,397 at the USPTO, along with all of the entabulated and listed patient and patent application of Applicant. In short, according to the instant teachings systems were patented from at least as early as December of 2020 for autonomous systems functioning based solely upon a priming dose associated with user's bodyweight with a learning algorithm on a patch.

Patch Pumps' transmission of adapted parameters is controlled by various triggers SEE for example BETA BIONICS' U.S. Pat. Nos. 11,594,313; 11,688,501 and 11,610,661. User interactions keep patches up to date and infusion algorithms constantly improving. FIG. 1-3 depicting myriad options such as 2 patches 2 cannulae to Boolean aggregates therefrom based in part on a first option being 2 sites, 2 cannulae and steel or Teflon/PTFE.

RPatch Pump systems (PPs) are defined herein as at least one of the iLET BIONIC PANCREAS Patch Pump system, the iLET BIONIC PANCREAS Patch Pump Bihormonal system and the iLET BIONIC PANCREAS Patch Pump system, a tubeless ACE pump, there are shown a bottom adhesive section for housing at least one of a steel cannulae, teflon (PTFE) cannulae, micro-needle arrays to provide ingress to the patient/user. As is known, these new infusion pumps can be small, light-weight and easy to use. Such Patch pumps stand as improvements in certain clinical settings, but have enumerated strong needs to improve fluid driving mechanisms from reservoirs through transcutaneous access tools—the present inventors hereby offer for consideration one set of such systems, as expressly labeled as desiderata by industry, including over the INSULET OMNIPOD a brand of device (see U.S. Pat. No. 10,420,883 Column 2, line 2). Highly controverted and the subject of ongoing patent litigations, the need for improvements is only underscored by the antiquated and longstanding mechanical challenges called out by these needs. Accordingly, even in germinal stages such modifications are embraced by and soon to become new standards of care in the industry, once again qualifying as progress in sciences and the useful arts, it is respectfully proposed and offered for consideration herein.

In short, among other things, the present inventors have created a system that supplants the strict need for any of these ‘clutch-types of mechanisms’ to drive fluid from pumps for delivering medicaments to patients with any and all manners of pumps, used for example, to deliver therapy for diabetes. This extends from conventional systems to patch pumps and later developed technologies. To make this patent, both the evolution of said pumps, and the instant improvements are explained herein, to connect the improvements to the historical developments, it is hereby earnestly solicited that such step changes are patentable as new, novel and non-obvious.

Driving the prior art was the fact that by using a clutch mechanism, the engagement between the leadscrew and the nut occurs at assembly, and thus no rotation is needed for the nut to engage the leadscrew by operation of the device. This reduces the number of fluid path prime pulses to prime the pump and assures a full and proper priming of the fluid path before placement on the body. The clutch mechanism also enabled the changing of thread pitch for other drug applications without a need to redesign the tilt nut used in fluid driving mechanisms in other existing pumps.

With the instant inventions, from priming methods through all types of fluid delivery, with both disposable and durable systems power management is improved enabling both rechargeable non-rechargeable paradigms.

### Novel and Enhanced of Glucose Control Agents

Until recently, technology limitations prevented the practical use of glucagon in
ambulatory medical devices. Native glucagon is highly unstable in aqueous solutions. It aggregates
and denatures within hours of reconstitution. As a result, glucagon previously needed to be administered quickly after reconstitution. It could not be used in automated systems, such as
ambulatory medical devices, because the glucagon would degrade when required. Recent
advancements include a non-aqueous, liquid-stable glucagon formulation developed by Xeris
Pharmaceuticals. Other advancements include Dasiglucagon, developed by Zealand
Pharmaceutical, which is a synthetic glucagon analog modified to be more stable in aqueous
solution than native glucagon.

The Xeris formulation uses dimethyl sulfoxide (DMSO) or similar solvents to suspend
glucagon in a non-aqueous matrix. This formulation prevents hydrolytic and conformational
degradation, resulting in a stable solution. The Xeris formulation can be packaged into auto-injectors or pumps and remains stable at room temperature for extended periods, potentially years. Ambulatory medical devices allow subjects the freedom to treat themselves while being mobile. Self-managed medical treatment comes with inherent risks to the subject.

+An automated blood glucose control system may automatically provide insulin and/or a counter-regulatory agent (e.g., Glucagon) to a subject to help control the blood glucose level of the subject. Generally, a control algorithm is implemented by an automated blood glucose control system (BGCS) to determine when to deliver one or more glucose control agents and how much agent to provide to the subject. Further, the control algorithm may control both an ongoing or periodic delivery of insulin (e.g., a basal dose), and a correction bolus that may be provided to adjust a subject's blood glucose level to within a desired range. The control algorithm may use blood glucose level readings obtained from a sensor, such as a continuous glucose monitoring (CGM) sensor, that obtained automated blood glucose measurements from the subject. Moreover, in some cases, the control algorithm may deliver a bolus of insulin in response to an indication of a meal to be consumed or being consumed by the subject.

Insulin may be administered subcutaneously into blood of a subject. There is often a delay between when the insulin is provided and when the amount of insulin in the subject's blood plasma reaches maximum concentration. This amount of time may vary based on the type of insulin and on the physiology of the particular subject. For example, with a fast-acting insulin, it may take approximately 65 minutes for a bolus of insulin to reach maximum concentration in the blood plasma of the subject. For some other types of insulin, it may take anywhere from 3-5 hours to reach maximum concentration in the blood plasma of the subject. Accordingly, the blood glucose control system may implement a predictive algorithm that implements a bi-exponential pharmacokinetic (PK) model that models the accumulation of insulin doses in the blood plasma of the subject. The blood glucose control system may modify its predictions based on the type of insulin, one or more blood glucose readings, and/or characteristics of the subject.

In some cases, a subject may receive a manual bolus of insulin or medicament. For example, a user (e.g., healthcare provider, parent, or guardian) or subject may inject a dose of insulin into the subject. As another example, the user or subject may manually direct the automated blood glucose control system to provide a bolus of insulin to the subject.

It is generally undesirable to have too much insulin. An excess of insulin can lead to Hypoglycemia. As described above, it may take time for insulin to reach maximum concentration in the blood plasma of the subject. Thus, a blood glucose level reading from a sensor may not immediately, or even after a particular period of time, reflect the amount of insulin within a subject. Thus, a manual bolus of insulin may not be detected by the automated blood glucose control system. As a result, if the automated blood glucose control system is operating during delivery of a manual bolus, or is configured to operate on the subject prior to blood glucose level readings reflecting the effect of the manual bolus on the subject, the automated blood glucose control system may unnecessarily administer additional insulin to the subject potentially leading to hypoglycemia.

The present disclosure relates to an automated blood glucose control system configured to provide automatic delivery of glucose control therapy to a subject and receive information about manual glucose control therapy provided to the subject. Using the received information about the manual glucose therapy, the automated blood glucose control system can adjust the blood glucose control algorithm to account for the manual dosing of insulin (or counter-therapy agents). The manual glucose control therapy may be provided by injection therapy, or it may be provided by an insulin pump.

In some cases, the automated blood glucose control system may receive an indication of insulin or medicament to administer to a subject in place of an automatically calculated dose of insulin. For example, the automated blood glucose control system may receive an indication that a subject is consuming or will consume a meal. The indication may include a type of meal to be consumed (e.g., breakfast, lunch, or dinner) and an estimate of the quantity of food or carbohydrates to be consumed (e.g., less than usual, a usual amount, more than usual, 30-40 grams of carbohydrates, 45-60 grams of carbohydrates, etc.). Based on the indication, or meal announcement, the automated blood glucose control system may calculate an amount of insulin to administer to the subject. The calculation may be based on an insulin to carbohydrate ratio provided by a clinician and/or determined by the automated blood glucose control system. Moreover, the calculation may be based at least in part on a history of blood glucose level measurements for the subject when consuming particular meals.

The calculated amount of insulin for the meal announced by the user may be presented to the user. The user (e.g., the subject) may modify the amount of insulin to administer. For example, the user may determine that for the size meal the subject is consuming or planning to consume, more or less insulin should be administered. In such cases, the user may modify the calculated insulin dosage to match the user's determination of the amount of insulin to administer. In some cases, the automated blood glucose control system may modify its control algorithm based on the user's input. Thus, future meal announcements may result in a calculation of insulin that satisfies the subject's insulin needs and/or preferences.

For example, automated blood glucose control system can receive a meal announcement from a user responsive to the user interaction with the user interface. The meal announcement can correspond to an indication of a size of a meal consumed or to be consumed by the subject as discussed herein. The automated blood glucose control system can determine a meal bolus of insulin to administer to the subject based at least in part on the meal announcement. The meal bolus of insulin can correspond to an amount of insulin to administer to the subject to compensate for a change in blood glucose attributable to the meal. The automated blood glucose control system can output for display an indication of the meal bolus of insulin. The automated blood glucose control system can receive an indication of a requested modification to the meal bolus of insulin from the user. The automated blood glucose control system operate a control algorithm for automatic generation of an insulin dosing signal configured to operate the medicament pump to control blood glucose level in the subject based at least in part on a glucose level of the subject and the modification to the meal bolus of insulin.

In some cases, the indication of an amount of a manual bolus may be received by a user entering a numerical value (e.g., an amount of insulin, a number of carbohydrates, or another calculation) associated with administering insulin, which may be considered a specified gesture interaction required for entry of the manual bolus of medicament. In some cases, a specified gesture interaction required for entry of the manual bolus of medicament may be a sliding action or other movement on a touchscreen to confirm or initiate desired functions as discussed herein. As described above, the automated blood glucose control system may automatically-calculate a meal dose of insulin and present it to a user via a user interface where a user may enter the manual bolus information. At the time of making the meal announcement, the user may have an option to enter the manual bolus. The meal controller of the blood glucose pump can provide a recommendation against the manual entry if there is a prior history of online operation or a basis for making the recommendation.

The information may be received from a user via a user interface. This user interface may be provided by the automated blood glucose control system. Alternatively, or in addition, the user interface may be generated by another device, such as a laptop or desktop, a smartphone, a smartwatch, or any other computing device that can communicate via wired or wireless communication with the automated blood glucose control system. The information may include one or more of: an indication of delivery of a manual bolus (e.g., via injection therapy), an amount of the manual bolus, a type of the insulin (or other medicament), a time when the manual bolus was delivered, a general location that the manual bolus was administered to the subject (e.g., back, stomach, arm, leg, etc.), a reason for the manual bolus (e.g., a meal, a maintenance dose, a blood glucose level reading, in advance of exercise, etc.), and any other information that may be useable by the blood glucose control system in controlling the blood glucose level of the subject.

Advantageously, in certain embodiments, providing manual dosing information to the automated blood glucose control system can help the blood glucose control system maintain the blood glucose level of the subject within a desired range when the automated features of the blood glucose control system are active or operational. For example, if the automated blood glucose control system determines from a CGM sensor reading that a subject's blood glucose level is high, the automated blood glucose control system might normally administer a bolus of insulin. However, if the automated blood glucose control system receives an indication that a manual bolus of insulin was administered recently (e.g., within the past thirty minutes), the automated blood glucose control system may reduce or not administer a bolus of insulin, thereby preventing a hypoglycemic event and providing glycemic control. In some such cases, the automated blood glucose control system may continue monitoring the blood glucose level of the subject and may administer additional insulin at a later time if the blood glucose level readings do not reflect an expected blood glucose level based on the reported manual bolus of insulin.

In some cases, it may be unnecessary to receive an indication of the manual bolus because, for example, a user may cause the automated blood glucose control system to provide the manual bolus. In such cases, the automated blood glucose control system may track the amount of insulin delivered and the timing of the administering of the bolus. To track the manual bolus, the automated blood glucose control system may store the information associated with the manual bolus in a therapy log. Accordingly, when the automated blood glucose control system is operating in an automatic mode, the automated blood glucose control system can access the therapy log to determine whether any manual bolus were administered and, if so, the timing and amount of the manual bolus.

In some cases, the automated blood glucose control system may model the diminishing of insulin, or other medicament, in the blood plasma over time based on the information associated with the manual bolus. Modeling the diminishing of medicament over time may be used to estimate a future effect of the medicament previously administered. In some cases, the model may account for previously administered medicament by the automated blood glucose control system. Further, in some cases, the model may account for physiological characteristics of the subject, such as the subject's weight or an input parameter related to the subject's weight (e.g., body mass value, body mass index value). Moreover, the model may account for perfusion over time of the medicament bolus from a subcutaneous infusion site into the blood plasma of the subject. Further, the automated blood glucose control system may model an accumulation of insulin, model time course of activity of insulin, or model a finite rate of utilization of insulin.

Based on the model, the automated blood glucose control system may adjust the automated administering of insulin, or other medicament, when operating in an automatic mode to automatically generate an insulin dosing signal configured to operate the medicament pump to control blood glucose level. Further, the automated blood glucose control system may operate the administering of medicament (e.g., by controlling a medicament pump) based on a glucose level of the subject and the modeled concentration of medicament in the subject, which can include a time course of activity of the medicament in the subject due to a finite rate of utilization of the medicament as discussed herein.

In some cases, the automated blood glucose control system may confirm that the manual bolus was delivered to the subject. The confirmation may be determined based at least in part on whether blood glucose level readings by the CGM sensor match or are within a threshold level anticipated by the automated blood glucose control system based on the manual dosing information. Alternatively, or in addition, the automated blood glucose control system may request, via a user interface, that a user confirm that the manual bolus was delivered. In cases where the manual bolus in delivered by the automated blood glucose control system, a user may be requested to confirm the administering of the manual bolus by using a particular gesture or sequence of interactions with a user interface (e.g., a touchscreen) of the automated blood glucose control system or of a device (e.g., laptop or smartphone, etc.) that communicates with the automated blood glucose control system.

As previously described, in some cases, the information relating to the manual bolus may include an amount of insulin and a reason the manual bolus was administered (e.g., for a meal of a particular size). In some such cases, the automated blood glucose control system may determine an amount of insulin the automated blood glucose control system would administer in an automatic operating mode based on the manual dosing information if the manual bolus had not been supplied. If the automated blood glucose control system determines it would have supplied a different quantity of the medicament, and if the difference exceeds a threshold, the automated blood glucose control system may adjust a blood glucose control algorithm to account for the difference. For example, the automated blood glucose control system may change the operating setpoint or range of insulin the automated blood glucose control system attempts to maintain in the subject. As another example, the automated blood glucose control system may supplement the manual bolus with additional insulin to account for an under-administering of insulin or may reduce a subsequent dosage of insulin to account for an over-administering of insulin.

As previously indicated, the automated blood glucose control system may maintain a therapy log of manual insulin therapy. This therapy log may be maintained based on the use of the automated blood glucose control system to provide a manual bolus or based on information provided by the user based on manual administering of insulin (e.g., via injection). The manual boluses may be supplied when the automated blood glucose control system is not operating, is not operating in an automatic mode, or is not connected to the subject. Once the automated blood glucose control system is connected to the subject and is configured in automatic mode, the automated blood glucose control system may determine therapy, if any, to provide to the subject based on a combination of the therapy log and the glucose control algorithm implemented by the automated blood glucose control system.

The automated blood glucose control system may generate a dose control signal based on the determined therapy. This dose control signal may be supplied to a medicament pump, which may control delivery of the medicament (e.g., insulin) to the subject.

In some cases, a user may control whether the automated blood glucose control system is operating in a manual mode or an automatic mode by interacting with a user interface of the automated blood glucose control system or of a device that communicate with the automated blood glucose control system. The user interaction may include any type of user interaction with a user interface. For example, the user interaction may include interaction why physical buttons or interactions with a touchscreen including gestures or taps on the touchscreen.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes a method including: providing an option to a user to select between receiving medicament using a manual delivery component or an automated delivery system. The method also includes receiving, by the automated delivery system, subjective information regarding the activity or action that may alter the blood-glucose level. The method also includes receiving, by the manual delivery component, an amount of the medicament to be infused. The method also includes storing a time and the amount of medicament that is infused into the automated delivery system that controls blood glucose level. Other embodiments of this aspect include corresponding computer systems, apparatus, and computer programs recorded on one or more computer storage devices, each configured to perform the actions of the methods.

Implementations may include one or more of the following features. The method where the automated delivery system modifies medicament delivery based on the time and the amount of medicament that was received from either the manual delivery component or the automated delivery system. The method where the manual delivery component includes a keypad which allows the user to type in the dosage amount of the desired medicament. The method where providing the option to select is provided prior to a user performing the activity that may alter the blood-glucose level. The method where the activity that may alter the blood-glucose level includes of consuming food or exercising. The method where the subjective information regarding the activity of consuming food includes the approximate relative size of the food that is to be digested. The method where the approximate relative size of the food is compared to the recommended meal doses for the user, and depending on whether the approximate relative size is the same, larger, or smaller than the recommended doses, the model predictive control component is able to determine the actions that is required to regulate the glucose level of the blood. The method where the subjective information regarding the activity of exercising includes the intensity and the duration of the exercise. The method where the intensity and the duration of the exercise is compared to the recommended intensity and duration, and depending on whether it is the same, larger, or smaller than the recommended intensity and duration, the automated delivery system is able to determine the actions that are required to regulate the glucose level of the blood. Implementations of the described techniques may include hardware, a method or process, or computer software on a computer-accessible medium.

One general aspect includes a system having a medical device configured to provide an option to a user to select between receiving medicament using a manual delivery component or an automated delivery system. The system also includes automated delivery system configured to receive subjective information regarding the activity that may alter the blood-glucose level. The system also includes a manual delivery component configured to receive an amount of the medicament to be infused. The system also includes where the medical device storing a time and the amount of medicament that is infused into an automated delivery system that controls blood glucose levels. Other embodiments of this aspect include corresponding computer systems, apparatus, and computer programs recorded on one or more computer storage devices, each configured to perform the actions of the methods.

Upon utilizing an ambulatory medical device to request for a therapy change, users may have different preferences. Therefore, it is desirable for modern technology, especially ambulatory medical devices to be equipped with optionality features. These optionality features may fulfill the different preferences of the users and subjects. The optionality features may allow users to control the therapy changes more closely and may allow them to be more engaged with the medical assistance of the ambulatory medical device.

In order to fulfill the variety of preferences, an ambulatory medical device needs to provide options which allows the user to either manually request the amount of the desired medicament or choose an automated delivery system that automatically delivers the right amount of the medicament at the right time without further assistance. For the manual component, the user may personally input the desired amount on a keypad that is provided by the medical device. The medical device further confirms and delivers the requested medicament. After the medicament is infused through a manual delivery component, the data is stored into a model predicative control component which is further used to control and regulate the blood glucose level. However, if the user decides to use the automated delivery system, the user must provide subjective information regarding the activity or the action that may alter the blood-glucose level. For example, if the blood-glucose level changing activity is consuming food, the user must provide the time and the dosage amount of the food that is going to be digested. This information is tied to the automated delivery system, and the subjective information is further stored into a model predicative control component.

Embodiments described herein include an ambulatory medical device that has a keypad which allows a user to type in a dose of insulin or glucagon to be administered to a user. A user may wish to receive a single dose of insulin prior to consuming food and decide how much insulin need to be administered. In other embodiments, the user may choose to receive a burst of glucagon due to low blood sugar because of physical activities. Embodiments may include the options for manual inputs of medicament and automated delivery system of medicament. In various implementations, the automated delivery system of medicament is driven by the blood glucose level or related trends. Embodiments herein address a problem that may arise when the user has just received a manual dose and has switched on the automated delivery system. In such cases, the automated delivery system may be made aware of all manual medicament infusion amounts and the timing of such infusions. Accordingly, the manual delivery component may inform the automated delivery system upon delivering any medicament the type of medicament delivered, the amount of medicament and the timing of the medicament delivered. By having the above information, the automated delivery system may determine the amount of medicament that is the user's blood stream and adjust the automated delivery of medicament and the timing of the automated delivery. Accordingly, embodiments are directed to allows for a risk free or minimized transition from the manual delivery component and the automated delivery system.

Differences from other system may include that the manual delivery may be tied to an automated delivery system, the dose input from the user is then stored into a new algorithm for evaluating post-prandial glucose or a MPC algorithm (Model Predictive Control) instead of the meal delivery algorithm and is handled by the latter algorithm for evaluating post-prandia glucose or a known MPC algorithm. Other embodiments may include selection being able to have a relativistic algorithmically tuned value, such as shown in FIG. 1A and FIG. 1B. Other embodiments may include a post-prandial excursion value driven algorithm that includes a usual size meal or larger size meal or small size meal, and teach that, for example, a user enters a qualitative meal announcement or an algorithmically detected meal. The system initially gives a meal bolus which is conservative and based on rules of thumb. During the post-prandial period, the glucose excursions are monitored and the meal bolus associated with this qualitative announcement is increased or decreased if the glucose values or pattern is higher or lower than expected, including improved algorithmic derivations of these data sets and glucose values. Embodiments may include correlating the manual inputs to asking the user what the size of the meal was and learning how the insulin affects the user. Embodiments may include correlating the manual inputs to asking the user what activity the user performed and learning how the glucagon affects the user for a particular activity.

Meal Adaptation using glucose excursions is thus understood by artisans to be able to include specialized algorithms and source code and extends meal boluses for snacks across user interactions and allows for example entering of qualitative meals announcements or algorithm detected meals to be factored into delivered meal boluses calculated based on post-prandial meal excursions by monitoring glucose at fixed intervals focused on lowest to highest glucose levels and weighted criterion systems. Linking new predicted glucose and delivery information to mixes of at least three factors allows the system to effect auto-titration of parameters for example by evaluating post-prandial glucose relative to glucose targets.

### Terminology

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more computers or processors. The code modules may be stored in any type of non-transitory computer-readable medium or other computer storage device. Some or all the methods may be embodied in specialized computer hardware. Further, the computing system may include, be implemented as part of, or communicate with an automated blood glucose system, an ambulatory medicament system, or an ambulatory medical device.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks and modules described in connection with the embodiments disclosed herein can be implemented or performed by a machine, such as a processing unit or processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable device that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. A computing environment can include any type of computer system, including, but not limited to, a computer system based on a microprocessor, a mainframe computer, a digital signal processor, a portable computing device, a device controller, or a computational engine within an appliance, to name a few.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

Many variations and modifications may be made to the above-described embodiments, the elements of which are to be understood as being among other acceptable examples. All such modifications and variations are intended to be included herein within the scope of this disclosure.

In addition to reducing basal insulin delivery, Exercise Mode will also reduce the amount of correction insulin delivered by the iLet due to the increase in the CGM Target Glucose from a maximum of 130 mg/dl (the Higher target) during normal operation to 150 mg/dl. This is consistent with the consensus recommendation to increase the target glucose used by automated insulin delivery systems during exercise⁸ and will combine with reduction in basal insulin to reduce the amount of insulin-on-board during exercise. Maintaining the ability of the bionic pancreas algorithms to deliver insulin in response to hyperglycemia (albeit using a higher target) instead of turning off correction insulin entirely will mitigate risk of developing severe or prolonged hypoglycemia during exercise mode.

Finally, Exercise Mode will reduce the amount of insulin delivered in response to meal announcements from the 75% of the predicted amount of insulin required given during normal operation to 50% of the predicted amount of insulin required, a relative reduction of 33%. This is consistent with the consensus recommendation of a relative reduction of meal insulin by 25-80% depending on the timing of the meal⁸. Of note, meal announcements made during Exercise Mode will not be used for adaptation of the meal announcement. In addition, if Exercise Mode is initiated in the four hours following a meal announcement, that meal announcement will not be used for adaptation of the meal announcement.

The iLet Exercise Mode will provide an intermediate option between the two current options available to iLet users—using the iLet in its normal mode or disconnecting from the iLet completely and receiving no insulin during the period of disconnection. Upon cancellation or expiration of the set time-period, the iLet will revert to normal settings.

The design of the iLet Exercise Mode is similar to features present in other automated insulin delivery systems meant to be used during exercise, as shown in Table 1 below. Human factors (HF) validation testing for Exercise Mode will be included in the HF validation study for the Bionic Pancreas Patch Pump System.

Given the reliance on established literature for the development of the proposed iLet Exercise Mode and similarity to Exercise/Activity Mode settings across the industry, Beta Bionics believes software verification and human factors validation testing is sufficient to support a future 510(k) submission to implement this change.

### Example Patch Pump System Third iLet Dosing Decision Software: Therapy Restore

### Design

The Therapy Restore feature is designed to be used during activation of a replacement iLet ACE Pump and may be used within 30 days of the last available data upload to the Beta Bionics Cloud. The feature will only be available to users of the iLet App. When users pair their existing iLet App Account with their replacement iLet ACE Pump, they will be presented with the option to select Therapy Restore and restore from their last known point (date) or start over. They will also have the option to start the iLet without the use of Therapy Restore. If it has been more than 30 days since their last available data upload, users will not be presented with the Therapy Restore option.

Initializing insulin delivery will be the same for either the Therapy Restore option or starting over from factory settings. Users will be walked through standard device set up—including confirming their bodyweight. Users will be required to set up their CGM before setting up the replacement iLet ACE Pump with enough time to allow the CGM sensor to warm up. Just like with a new pump, the pump cannot be initialized until the CGM sensor is warmed up and displaying a glucose value.

Selecting Therapy Restore will restore the user's CGM Target setting, Secondary CGM Target setting (if utilized), bodyweight, and meal announcement adaptation (Usual for me Breakfast, Lunch, and Dinner). Users will be asked to confirm their bodyweight prior to initialization. Basal rate will not be restored, so basal insulin dosing will begin based on their body weight.

### Discussion

The Therapy Restore feature was designed in response to requests from users and HCPs asking for a feature that would allow return to steady state glucose control on the iLet as quickly as possible if the iLet had to be replaced for any reason. Although adaptation of the Basal and Correction algorithms occurs quickly, meal dose adaption may take up to one week because of the limited number of meals announced and therefore the limited number of opportunities to adapt the meal doses. Therapy Restore will initialize the iLet with the previously adapted meal doses to allow users to short-cut the part of adaptation that takes the longest.

In contrast to meal doses, adaptation of the basal and correction algorithms occur very rapidly. In the Bionic Pancreas Pivotal Trial participants randomized to the iLet arm reached their new time in range by 48 hours after initialization, on average. Most of the increase in time in range occurred in the first 24 hours. This is due to the speed of adaptation of the basal and correction algorithms. Therefore, it is unnecessary to restore the nominal basal profile or the aggressiveness parameter for the Correction algorithm to return quickly to the users'steady state glucose control. It is more conservative to return to the weight-based initialization settings for these algorithms, which are low for most users and therefore conservative, and that were validated in the Bionic Pancreas Pivotal Trial as being safe.

It is no less safe to restart the iLet using previously adapted meal doses than to start the iLet from factory settings and require the meal doses to adapt. We have chosen to limit the use of Therapy Restore to individuals with adapted meal doses that have been updated no longer than 30 days prior to starting the new iLet. This should ensure that there has been no significant changes in the insulin needs of the user since the meal doses were updated. Thirty days is one-third of the shortest typical interval between endocrinologist visits, which is the shortest interval between updates to the carbohydrate-to-insulin ratio and carbohydrate counting. In practice, the carbohydrate-to-insulin ratio is typically updated much less frequently than every three months, so a 30-day period to allow use of previously adapted meal doses is very conservative. In addition, the iLet gives 75% of the insulin predicted to be needed for a meal, providing a margin of safety.

If the user or the HCP is aware of significant change in the meal insulin need during the interval between the time the last update of iLet data to the cloud and the start of the new iLet, there is always the option to start the iLet using factory settings

1. An enhanced patch pump system comprised of at least a user friendly and easily managed set-up imparted with a plurality of animations, schematics and improved GUI's whereby a user may self-start the system without training.

2. The enhanced patch pump system of claim 1, further comprised of: at least a durable patch pump and disposable cartridge.

3. The enhanced patch pump system of claim 2, further comprised of:
at least a fill needle and syringe.

4. The enhanced patch pump system of claim 3, further comprised of: at least an alcohol preparation wipe assembly.

5. The enhanced patch pump system of claim 4, further comprised of: at least a vial of medicament.

6. The enhanced patch pump system of claim 5, being comprised of: at least one of a vial of insulin and a vial of glucagon-like moiety.

7. A method for patch pump set, which comprises, in combination: providing at least a durable patch pump and disposable cartridge.

8. The method for patch pump set according to claim 7, which comprises, in combination: assembling the syringe by twisting the fill needle clockwise onto the top of the syringe until tight.

9. The method for patch pump set according to claim 8, which comprises, in combination: preparing the syringe by removal of the needle cap.

10. The method for patch pump set according to claim 9, which comprises, in combination: pulling back on the plunger to draw air into the syringe equal to the amount of medicament to be used—for example between 25 units (0.2′ mL) and 200 units (2 mL).

11. The method for patch pump set according to claim 10, which comprises, in combination:
cleaning the vial top with an alcohol wipe, inserting the needle and pushing the plunger to insert the air; and flipping the vial and syringe and pulling the plunger to fill the syringe.

12. The method for patch pump set up according to claim 11, which comprises, in combination:
checking for air bubbles in the syringe, and and flicking them to the top if there are any while pushing the plunger to remove any air subject air bubbles.

13. The method for patch pump set up according to claim 12, which comprises, in combination:
filling a cartridge by leaving the cartridge in a tray, inserting the needle straight into the fill port while slowly pushing down on the plunger to fill the cartridge, and removing the needle when done.

14. The method for patch pump set up according to claim 13, which comprises, in combination:
at least one of scanning a QR Code on the inside of the durable pump, and manually entering a QR code being also located on the side of an associated box;
assembling the durable pump and disposable patch assembly by sliding said pump and filled cartridge together until an audible click occurs.

15. The method for patch pump set up according to claim 14, which comprises, in combination:
pump priming, being beeping while said pump is priming;
once beeping is stopped, cleaning the insertion site with alcohol wipes;
removing the pull tab and liner, as while removing the pull tab, the attached liner will be removed with it, then removing the remaining adhesive liner, completing the set up.

16. The enhanced patch pump system of claim 1, used with at least a first drive nut having a length extending longitudinally along the pump housing, the first drive nut extending into the first medicament cartridge receptacle via the first aperture and in communication with the first lead screw, the first drive nut being configured to urge forward or backward in response to a first direction of rotation and a second direction of rotation, respectively, of a first lead screw.

17. The enhanced patch pump system of claim 2, whereby once the first drive nut is past a point it becomes locked into the rotation system, and a tooth engages the nut and stops it from locking.

18. The enhanced patch pump system of claim 17, further comprising a plunger staying until actuated by a trigger switch, being a contact switch, causing travel to the interface.

19. The enhanced patch pump system of claim 18, further comprising so determining the insulin fill, or glucagon-like moiety fill, and not using a traditional clutch type of mechanism.

20. The enhanced patch pump system of claim 19, further comprising a first seal, the first seal providing a water resistant barrier between the internal area of the infusion pump and the first medicament cartridge receptacle; wherein the first seal is a first O-ring that surrounds at least a portion of the first drive nut, wherein the pump housing comprises a first saddle, the first saddle being configured to retain the first O-ring.
